(19) United States
(12) Patent Application Publication
(10) Pub. No.: US 2008/0176902 A1
(43) Pub. Date: Jul. 24, 2008

(54) Salt III

(75) Inventors: Julien Giovannini, Loughborough (GB); Bo-Goran Josefsson, Lund (SE)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/997,489

(22) PCT Filed: Jul. 31, 2006
(86) PCT No.: PCT/SE2006/000922

§ 371 Date: Jan. 31, 2008

(30) Foreign Application Priority Data
Aug. 2, 2005 (SE) 0501769-4

(51) Int. Cl. A61K 31/445; C07D 211/56; A61P 11/06

(52) U.S. Cl. 514/329; 546/223

(57) ABSTRACT

The invention provides N-{2-[((2S)-3-{[1-(4-chlorobenzyl)piperidin-4-yl]amino}-2-hydroxy-2-methyl-propyl)oxy]-4-hydroxyphenyl}acetamide hemi-fumarate or a solvate thereof, pharmaceutical compositions containing the salt or solvate and use of the salt or solvate in therapy.

The present invention relates to a salt of a piperidine derivative, pharmaceutical composition containing it and its use in therapy.

Chemokine Receptor 1 (CCR1) is highly expressed in tissues affected in different autoimmune, inflammatory, proliferative, hyper proliferative and immunologically mediated diseases, e.g. asthma, chronic obstructive pulmonary disease, multiple sclerosis and rheumatoid arthritis. Therefore, inhibiting CCR1-mediated events with the salt of the invention, e.g. by cell activation and migration, is expected to be effective in the treatment of such conditions.

In the manufacture of pharmaceutical formulations, it is important that the active compound is in a form in which it can be conveniently handled and processed in order to obtain a commercially-viable manufacturing process. In this connection, the chemical stability and the physical stability of the active compound are important factors. The active compound, and formulations containing it, must be capable of being effectively stored over appreciable periods of time, without exhibiting any significant change in the physico-chemical characteristics (e.g. chemical composition, density, hygroscopicity and solubility) of the active compound.

Furthermore, if the active compound is to be incorporated into a formulation for pulmonary administration, e.g., via a dry powder inhaler such as the Turbuhaler® device, it is desirable if the active compound can be readily micronised to yield a powder with good flow properties and comprising a high fine crystalline particle fraction (i.e. a fraction in which the active compound particles have a mass median aerodynamic diameter of less than 10 μm (micrometer)). Such a fraction is capable of being carried deep into the lungs leading to faster and increased absorption of the active compound.

International Patent Application Publication No. WO 03/051839 generally discloses certain piperidinyl derivatives that have activity as CCR1 antagonists and, in particular, the compound 4-({(2S)-3-[2-(acetylamino)-5-hydroxyphenoxy]-2-hydroxy-2-methylpropyl}ammonio)-1-(4-chlorobenzyl)piperidine and pharmaceutically acceptable salts or solvates thereof. The only salt of this compound specifically disclosed in the application is the ditrifluoroacetate salt, which being amorphous in character, does not make it suitable for use in a dry powder formulation for pulmonary administration.

It has now surprisingly been found possible to prepare a salt of the compound 4-({(2S)-3-[2-(acetylamino)-5-hydroxyphenoxy]-2-hydroxy-2-methylpropyl}ammonio)-1-(4-chlorobenzyl)piperidine having good physico-chemical properties which is capable of being formulated in a dry powder formulation for pulmonary administration.

The structure of 4-({(2S)-3-[2-(acetylamino)-5-hydroxyphenoxy]-2-hydroxy-2-methylpropyl}ammonio)-1-(4-chlorobenzyl)piperidine is shown below:

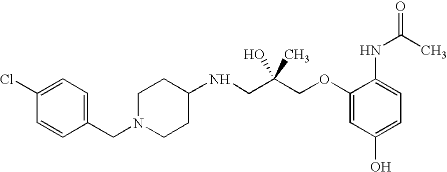

Thus, in accordance with the present invention, there is provided the hemi-fumarate salt of 4-({(2S)-3-[2-(acetylamino)-5-hydroxyphenoxy]-2-hydroxy-2-methylpropyl}ammonio)-1-(4-chlorobenzyl)piperidine (hereinafter referred to as N-{2-[((2S)-3-{[1-(4-chlorobenzyl)piperidin-4-yl]amino}-2-hydroxy-2-methylpropyl)oxy]-4-hydroxyphenyl}acetamide hemi-fumarate, “the hemi-fumarate salt”).

The invention also provides solvates (including hydrates) of the hemi-fumarate salt. However, the hemi-fumarate salt is preferably anhydrous, and preferably in non-solvated form.

In an embodiment of the invention, the hemi-fumarate salt or solvate thereof has crystalline properties and is preferably at least 50% crystalline, more preferably at least 60% crystalline, still more preferably at least 70% crystalline and most preferably at least 80% crystalline. Crystallinity can be estimated by conventional X-ray diffractometry techniques.

In another embodiment of the invention, the hemi-fumarate salt or solvate thereof is from 50%, 60%, 70%, 80% or 90% to 95%, 96%, 97%, 98%, 99% or 100% crystalline.

The hemi-fumarate salt exhibits at least the following characteristic X-ray powder diffraction (XRPD) peaks (expressed in degrees 20) (the margin of error being consistent with the United States Pharmacopeia general chapter on X-ray diffraction (USP941)—see the United States Pharmacopeia Convention. X-Ray Diffraction, General Test <941>. United States Pharmacopeia, 25th ed. Rockville, Md.: United States Pharmacopeial Convention; 2002:2088-2089):

(1) 6.2, 10.7 and 12.5, or
(2) 6.2, 10.7 and 18.8, or
(3) 6.2, 10.7 and 18.0, or
(4) 6.2, 10.7, 12.5, 18.0 and 18.8, or
(5) 6.2, 10.7, 12.5, 18.0, 18.8, 19.7 and 19.8.

The hemi-fumarate salt may be prepared by a process comprising the following steps:
(i) forming a reaction mixture by contacting, preferably with stirring, N-{2-[((2S)-3-{[1-(4-chlorobenzyl)piperidin-4-yl]amino}-2-hydroxy-2-methylpropyl)oxy]-4-hydroxyphenyl}acetamide with fumaric acid in the presence of a suitable solvent or mixture of solvents (e.g. an organic solvent such as a polar solvent, examples of which include methanol, ethanol, n-propanol, isopropanol, acetone and ethyl acetate),
(ii) obtaining a precipitate of N-{2-[((2S)-3-{[1-(4-chlorobenzyl)piperidin-4-yl]amino}-2-hydroxy-2-methylpropyl)oxy]-4-hydroxyphenyl}acetamide hemi-fumarate, and
(iii) separating the precipitate from the reaction mixture.

The compounds of the invention are useful as modulators of CCR1 or MIP-1α chemokine receptor activity [N-{2-[((2S)-3-{[1-(4-chlorobenzyl)piperidin-4-yl]amino}-2-hydroxy-2-methylpropyl)oxy]-4-hydroxyphenyl}acetamide ditrifluoroacetate has an IC50 of below 50 nM in the Human CCR1 binding assay described in the Example section herein] and may be administered to a mammal, including man, for the treatment of autoimmune, inflammatory, proliferative and hyperproliferative diseases and immunologically-mediated diseases.

Examples of these conditions are:

1. respiratory tract: obstructive diseases of the airways including: asthma, including bronchial, allergic, intrinsic, extrinsic, exercise-induced, drug-induced (including aspirin and NSAID-induced) asthma, chronic or inverterate asthma (e.g. late asthma and airways hyper-responsiveness), and dust-induced asthma, both intermittent and persistent and of all severities, and other causes of airway hyper-responsiveness; chronic obstructive pulmonary disease (COPD), such as irreversible COPD; bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute, allergic, atropic rhinitis and chronic rhinitis including rhinitis caseosa, hypertrophic rhinitis, rhinitis purulenta, rhinitis sicca and rhinitis medicamentosa, and vasomotor rhinitis; membranous rhinitis including croupous, fibrinous and pseudomembranous rhinitis and scrofoulous rhinitis; perennial and seasonal (allergic) rhinitis including rhinitis nervosa (hay fever); nasal polyposis; acute viral infection including the common cold, and infection due to respiratory syncytial virus, influenza, coronavirus (including SARS) and adenovirus;

2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthropathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositis and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies; and Reiter's disease;

3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);

4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions; bullous Pemphigus; uveitis and vernal conjunctivitis;

5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;

6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);

7. abdominal: hepatitis, including autoimmune, alcoholic and viral; fibrosis and cirrhosis of the liver; cholecystitis; pancreatitis, both acute and chronic;

8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);

9. allograft rejection: acute and chronic following, for example, transplantation of kidney, heart, liver, lung, bone marrow, skin or cornea or following blood transfusion; or chronic graft versus host disease;

10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;

11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;

12. other disorders with an inflammatory or immunological component; including acquired immune deficiency syndrome (AIDS), leprosy, Sezary syndrome, and paraneoplastic syndromes; systemic lupus, erythematosus; lepromatosous leprosy; type I diabetes, nephrotic syndrome;

13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;

14. oncology: treatment of common cancers including prostate, breast, lung (e.g. non-small cell lung cancer (NSCLC), ovarian, pancreatic, bowel and colon, stomach, skin and brain tumors, and squamous sarcoma, and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,

15. gastrointestinal tract: Coeliac disease, proctitis, eosinophilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

Thus, the present invention provides N-{2-[((2S)-3-{[1-(4-chlorobenzyl)piperidin-4-yl]amino}-2-hydroxy-2-methylpropyl)oxy]-4-hydroxyphenyl}acetamide hemi-fumarate or a solvate thereof for use in therapy.

In a further aspect, the present invention provides the use of N-{2-[((2S)-3-{[1-(4-chlorobenzyl)piperidin-4-yl]amino}-2-hydroxy-2-methylpropyl)oxy]-4-hydroxyphenyl}acetamide hemi-fumarate or a solvate thereof in the manufacture of a medicament for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention also provides a method of treating an inflammatory disease in a patient suffering from, or at risk of, said disease, which comprises administering to the patient a therapeutically effective amount of N-{2-[((2S)-3-{[1-(4-chlorobenzyl)piperidin-4-yl]amino}-2-hydroxy-2-methylpropyl)oxy]-4-hydroxyphenyl}acetamide hemi-fumarate or a solvate thereof.

The invention still further provides a method of treating an airways disease, e.g. a reversible obstructive airways disease, in a patient suffering from, or at risk of, said disease, which comprises administering to the patient a therapeutically effective amount of N-{2-[((2S)-3-{[1-(4-chlorobenzyl)piperidin-4-yl]amino}-2-hydroxy-2-methylpropyl)oxy]-4-hydroxyphenyl}acetamide hemi-fumarate or a solvate thereof.

For the above-mentioned therapeutic uses the dosage administered will, of course, vary with the mode of administration, the treatment desired and the disorder indicated but will typically be in the range from 0.001 mg/kg to 30 mg/kg.

The hemi-fumarate salt or solvate thereof according to the invention may be used on its own but will generally be administered in the form of a pharmaceutical composition in which the hemi-fumarate salt or solvate thereof (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition may comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising N-{2-[((2S)-3-{[1-(4-chlorobenzyl)piperidin-4-yl]amino}-2-hydroxy-2-methylpropyl)oxy]-4-hydroxyphenyl}acetamide hemi-fumarate or a solvate thereof in association with a pharmaceutically acceptable adjuvant, diluent or carrier.

The invention further provides a process for the preparation of a pharmaceutical composition of the invention which comprises mixing N-{2-[((2S)-3-{[1-(4-chlorobenzyl)piperidin-4-yl]amino}-2-hydroxy-2-methylpropyl)oxy]-4-hydroxyphenyl}acetamide hemi-fumarate or a solvate thereof with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

In an embodiment of the invention, the hemi-fumarate salt of the invention is administered by inhalation. In a further embodiment, the hemi-fumarate salt of the invention is administered by means of a dry powder inhaler. The inhaler may be a single or a multi dose inhaler, and may be a breath actuated dry powder inhaler.

When administered via inhalation the dose of the compound (i.e. hemi-fumarate salt) of the invention may generally be in the range of from 0.1 μg to 10000 μg, 0.1 to 5000 μg, 0.1 to 1000 μg, 0.1 to 500 μg, 0.1 to 200 μg, 0.1 to 200 μg, 0.1 to 100 μg, 0.1 to 50 μg, 5 μg to 5000 μg, 5 to 1000 μg, 5 to 500 μg, 5 to 200 μg, 5 to 100 μg, 5 to 50 g, 10 to 5000 μg, 10 to 1000 μg, 10 to 500 μg, 10 to 200 μg, 10 to 100 μg, 10 to 50 μg, 20 to 5000 μg, 20 to 1000 μg, 20 to 500 μg, 20 to 200 μg, 20 to 100 μg, 20 to 50 μg, 50 to 5000 μg, 50 to 1000 μg, 50 to 500 μg, 50 to 200 μg, 50 to 100 μg, 100 to 5000 μg, 100 to 1000 μg or 100 to 500 μg.

Dry powder formulations and pressurized HFA aerosols of the compounds of the invention may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

The invention therefore further relates to combination therapies wherein a compound of the invention or a pharmaceutical composition or formulation comprising a compound of the invention is administered concurrently or sequentially or as a combined preparation with another therapeutic agent or agents, for the treatment of one or more of the conditions listed.

The present invention will now be further explained by reference to the following illustrative examples.

### General Methods

¹H NMR spectra were recorded at 298K on a Varian Unity Inova 400 MHz (software: VNMR 6.1C and VNMRJ 1.1D; probe: Nalorac 5 mm DG400-5AT) or a Varian Mercury-VX 300 MHz (software: VNMR 6.1C; probe: Varian 5 mm AutoSW PFG) instrument. The central peaks of acetone-d₆ or dimethylsulphoxide (DMSO)-d₆ were used as internal references.

The following method was used for LC/MS analysis:

| | |
|---|---|
| MS Instrument: | Agilent 1100 series, equipped with APCI interface |
| LC instrument: | Agilent 1100 series, equipped with UV-detector |
| | VWD, autosampler ALS, binary pump and degasser |
| LC-column: | Chromolith Speed ROD, RP-C18, ø 4.6 × 50 mm |
| Eluant: | Solvent A: water + 0.1% trifluoroacetic acid |
| | (TFA); Solvent B: acetonitrile + 0.1% TFA |
| Conditions | LC: flow 2.5 ml/minute; 5 to 95% B in gradient; |
| | run time 3.6 minutes; UV 220 nm |
| | MS: positive detection; capillary voltage 3 kV |

### EXAMPLE 1

### Preparation of N-{2-[((2S)-3-{[1-(4-chlorobenzyl)piperidin-4-yl]amino}-2-hydroxy-2-methylpropyl)oxy]-4-hydroxyphenyl}acetamide hemi-fumarate (2:1 salt)

To a stirred solution of crude N-{2-[((2S)-3-{[1-(4-chlorobenzyl)piperidin-4-yl]amino}-2-hydroxy-2-methylpropyl)oxy]-4-hydroxyphenyl}acetamide (24.0 g, 36.5 mmol; obtained by extraction at pH 9 from the corresponding salt with trifluoroacetic acid as described in Example 1 of WO 03/051839) in methanol (240 ml), a solution of fumaric acid (2.13 g, 18.3 mmol) in methanol (55 ml) was added over a period of 15 minutes. It was observed that a precipitate began to form when about two thirds of the fumaric acid solution had been added. When all the fumaric acid solution had been added, the stirring was stopped and the reaction mixture was left overnight at ambient temperature (20° C.) in a closed flask. The precipitate was isolated on a filter funnel, washed with methanol (3×50 ml) and dried in vacuo at 60° C. overnight to give the titled salt as an off-white solid (14.0 g, 73%).

¹H NMR (399.99 MHz, DMSO-d₆) δ 8.91 (s, 1H), 7.48 (d, J=8.6 Hz, 1H), 7.38 (d, J=8.5 Hz, 2H), 7.31 (d, J=8.4 Hz, 2H), 6.50 (s, 1H), 6.42 (d, J=2.5 Hz, 1H), 6.31 (dd, J=8.6, 2.5 Hz, 1H), 3.79 (s, strongly coupled AB-system, 2H), 3.44 (s, 2H), 2.88 (d, J=12.2 Hz, 1H), 2.82-2.72 (m, 3H), 2.64-2.55 (m, 1H), 2.02 (s, 3H), 2.00-1.92 (m, 2H), 1.91-1.83 (m, 2H), 1.47-1.35 (m, 2H), 1.23 (s, 3H)

APCI-MS: m/z 462 [MH+]

The stoichiometry, base to acid, of 2:1 was confirmed by NMR.

### EXAMPLE 2

### X-Ray Powder Diffraction Analyses

### General Procedures

X-ray powder diffraction (XRPD) analyses may be performed on samples prepared according to standard methods (see for example Giacovazzo et al., eds., Fundamentals of Crystallography, Oxford University Press (1992); Jenkins & Snyder, eds., Introduction to X-Ray Powder Diffractometry, John Wiley & Sons, New York (1996); Bunn, ed., Chemical Crystallography, Clarendon Press, London (1948); and Klug & Alexander eds., X-ray Diffraction Procedures, John Wiley & Sons, New York (1974)).

An X-ray powder diffraction pattern of the hemi-fumarate salt described in Example 1 above (in anhydrous form) was obtained as described below:

A Bragg-Brentano parafocusing powder X-ray diffractometer using monochromatic CuKα radiation (45 kV and 40 mA) was used for the analyses. The primary optics contained soller slits and an automatic divergence slit. Flat samples were prepared on zero background plates that were rotated during the measurements. The secondary optics contained soller slits, an automatic anti scatter slit, a receiving slit and a monochromator. The diffracted signal was detected with a proportional xenon-filled detector. Diffraction patterns were collected between 2°≦2θ (theta)≦40° in a continuous scan mode with a step size of 0.016° 2θ at a rate of 4° 2θ per minute. Raw data were stored electronically. Evaluation was performed on raw or smoothed diffraction patterns.

A Panalytical X'pert PRO MPD θ-θ diffractometer in reflection mode was used for the above-mentioned measurements. A person skilled in the art can set up instrumental parameters for a powder X-ray diffractometer so that diffraction data comparable to the data presented can be collected.

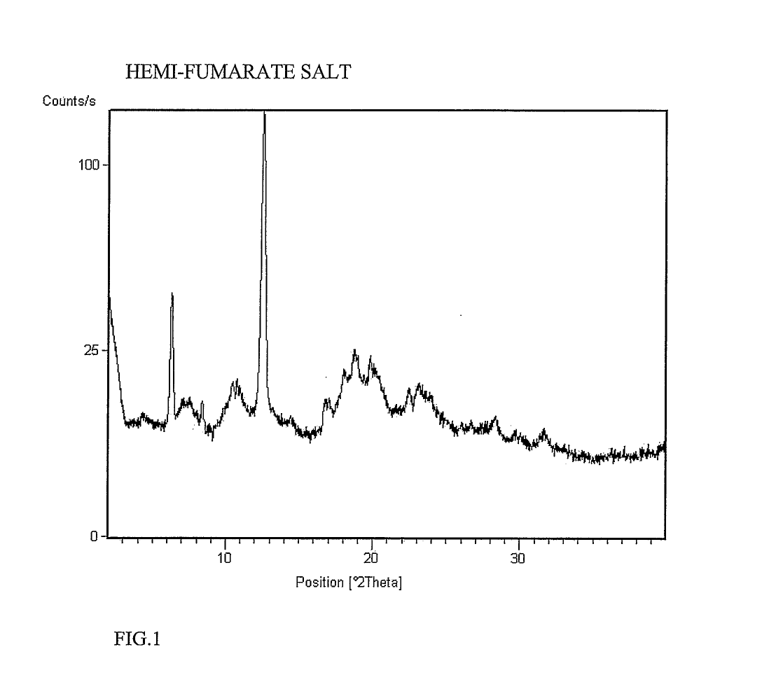

The results obtained are shown in FIG. 1.

### EXAMPLE 3

### Differential Scanning Calorimetry (DSC)

Using standard methods, for example those described in Höhne, G. W. H. et al (1996), Differential Scanning Calorimetry, Springer, Berlin, the calorimetric response of a test sample to increasing temperature was investigated using a Q1000 Modulated Temperature Differential Scanning Calorimeter (MTDSC) in “heat only” mode with a ramp rate of 5° C. per minute. Approximately 2 to 5 mg of test sample was placed in aluminium cups with lids (no crimping) under a nitrogen atmosphere.

It is well known that the DSC onset and peak temperatures may vary due to the purity of the sample and instrumental parameters, especially the temperature scan rate. A person skilled in the art can set up instrumental parameters for a differential scanning calorimeter so that data comparable to the data presented here can be collected.

The melting temperature for a typical sample of the hemi-fumarate salt described in Example 1 above was found to be 189° C.±2° C. (onset).

### Human CCR1 Binding Assay

### Membranes

HEK293 cells, from ECACC, stably expressing recombinant human CCR1 (HEK-CCR1) were used to prepare cell membranes containing CCR1. The membranes were stored at −70° C. The concentration of membranes of each batch was adjusted to 10% specific binding of 33 pM [¹²⁵I] MIP-1α.

### Binding Assay

100 μL of HEK-CCR1 membranes diluted in assay buffer pH 7.4 (137 mM NaCl (Merck, Cat No 1.06404), 5.7 mM Glucose (Sigma, Cat No G5400), 2.7 mM KCl (Sigma, Cat No P-9333), 0.36 mM NaH₂PO₄×H₂O (Merck, Cat No 1.06346), 10 mM HEPES (Sigma, Cat No H3375), 0.1% (w/v) Gelatine (Sigma, Cat No G2625)) with the addition of 17500 units/L Bacitracin (Sigma, Cat No B1025) were added to each well of the 96 well filter plate (0.45 μm opaque Millipore cat no MHVB N4550). 12 μL of compound in assay buffer, containing 10% DMSO, was added to give final compound concentrations of 1×10^{-5.5}-1×10^{9.5} M. 12 μl cold human recombinant MIP-1α (270-LD-050, R&D Systems, Oxford, UK), 10 nM final concentration in assay buffer supplemented with 10% DMSO, was included in certain wells (without compound) as non-specific binding control (NSB). 12 μl assay buffer with 10% DMSO was added to certain wells (without compound) to detect maximal binding (B0).

12 μL [¹²⁵I] MIP-1α, diluted in assay buffer to a final concentration in the wells of 33 pM, was added to all wells. The plates with lid were then incubated for 1.5 hrs at room temperature. After incubation the wells were emptied by vacuum filtration (MultiScreen Resist Vacuum Manifold system, Millipore) and washed once with 200 μl assay buffer. After the wash, all wells received an addition of 50 μL of scintillation fluid (OptiPhase “Supermix”, Wallac Oy, Turko, Finland). Bound [¹²⁵I] MIP-1: was measured using a Wallac Trilux 1450 MicroBeta counter. Window settings: Low 5-High 1020, 1-minute counting/well.

### Calculation of Percent Displacement and IC₅₀

The following equation was used to calculate percent displacement.

Percent displacement=1−((cpm test−cpm NSB)/(cpm B0−cpm NSB)) where:

cpm test=average cpm in duplicate wells with membranes and compound and [¹²⁵I] MIP-1α cpm;
NSB=average cpm in the wells with membranes and MIP-1α and [¹²⁵I] MIP-1α (non-specific binding) cpm;
B0=average cpm in wells with membranes and assay buffer and [¹²⁵I] MIP-1α (maximum binding).

The molar concentration of compound producing 50% displacement (IC₅₀) was derived using the Excel-based program XLfit (version 2.0.9) to fit data to a 4-parameter logistics function.

1. A compound being N-{2-[((2S)-3-{[1-(4-chlorobenzyl)piperidin-4-yl]amino}-2-hydroxy-2-methylpropyl)oxy]-4-hydroxyphenyl}acetamide hemi-fumarate or a solvate thereof.

2. A compound according to claim 1 which is anhydrous.

3. A compound according to claim 2 which exhibits at least the following characteristic X-ray powder diffraction peaks (expressed in degrees 2θ):
(1) 6.2, 10.7 and 12.5, or
(2) 6.2, 10.7 and 18.8, or
(3) 6.2, 10.7 and 18.0, or
(4) 6.2, 10.7, 12.5, 18.0 and 18.8, or
(5) 6.2, 10.7, 12.5, 18.0, 18.8, 19.7 and 19.8.

4. A compound according to claim 3 having an X-ray powder diffraction pattern substantially the same as that shown in FIG. 1.

5. A pharmaceutical composition comprising a compound according to claim 1 in association with a pharmaceutically acceptable adjuvant, diluent or carrier.

6. A compound according to claim 1 or a pharmaceutical composition according to claim 5 in combination with a dry powder inhaler.

7-10. (canceled)

11. A method of treating an airways disease in a patient suffering from, or at risk of, said disease, which comprises administering to the patient a therapeutically effective amount of a compound according to claim 1 or a pharmaceutical composition according to claim 5.

12. The method of claim 11, wherein the airways disease is chronic obstructive pulmonary disease.

13. The method of claim 11, wherein the airways disease is asthma.

14. A method for the treatment of a human disease or condition in which modulation of chemokine receptor 1 (CCR1) activity is beneficial, which comprises administering to a patient suffering from said disease or condition a therapeutically effective amount of a compound according to claim 1 or a pharmaceutical composition according to claim 5.
